(19) United States
(12) Patent Application Publication
(10) Pub. No.: US 2005/0033121 A1
(43) Pub. Date: Feb. 10, 2005

(54) Diagnostic information systems

(76) Inventor: Ivan E. Modrovich, Camarillo, CA (US)

Correspondence Address: BACON & THOMAS, PLLC, 625 SLATERS LANE, FOURTH FLOOR, ALEXANDRIA, VA, 22314, US

(21) Appl. No.: 10/499,642

(22) PCT Filed: Dec. 30, 2002
(86) PCT No.: PCT/US02/38986

Related U.S. Application Data
(60) Provisional application No. 60/343,333, filed on Dec. 28, 2001.

(51) Int. Cl. A61B 5/00

(52) U.S. Cl. 600/300

(57) ABSTRACT

Relevant clinician determined diagnostic and marker information forming a patients profile are fed into a computer system containing comparative profiles. The comparative results are reported to the clinician as are recommendations for treatment and further investigation if desired. A final diagnosis is reported and treatment, if utilized, is fed back to enhance the computerized profiles.

### CROSS REFERENCE TO RELATED APPLICATIONS

This is a completed specification of the Invention disclosed in Provisional Application Ser. No. 60/343,333 filed Dec. 28, 2001, the benefit of the filing date of which is claimed.

### BACKGROUND OF THE INVENTION

Diagnostic methods of diseases and other malady detention by a clinician, i.e. a physician or others under the physician's direction and control, rely on the identification and evaluation of quantifable markers, and other information. Markers include such things as risk factors, indicators based on family history, demographics and environmental conditions, quantifiable signs and symptoms, and analytes found in biological fluids, such as blood. Currently, the diagnosis of a dive or other malady relies upon the subjective analysis of markers collected by a clinician. Unfortunately, this subjective analysis process most often cannot review and evaluate all the critical and relevant actors and give appropriate level of weighting in order to reach an accurate and timely diagnosis.

While the collective knowledge base of diagnostic information is vast, and the medical profession still relies heavily on yesterday's technology of organizing and providing the medical profession with information primarily through publications. In diagnosing a patient's health or disease state, the physician is largely relegated to searching the literature, consulting specialists, and relying on personal knowledge and experience.

The current and growing trend in disease diagnosis is to utilize information based on an exchange between patients and computers. It lacks, in good part, the control of the medical professional.

U.S. Pat. No. 6,196,970 to Brown discloses a method whereby data is collected from a plurality of research subjects and used to update a research protocol. While a medical research expert is used to determine any changes to the protocol, the information provided is controlled by the research subject and therefore limited by the accuracy of the subject's answers. No requirement is made of the subject's health or lack thereof.

U.S. Pat. No. 6,270,456 to Iliff pertains to a system and method whereby the patient imparts information to a computer using a list contained in the computer or using existing computer based diagnostic scripts. The computer controls the information and is limited by information input by the patient. Responses to questions are analyzed and converted into symptoms which are compared to symptoms on file. This system is limited by the computer and the patient.

U.S. Pat. No. 6,247,004 to Moukbeibir against which there was cited some 34 patents and 10 publications, is directed to a computer system that determines possible events of a large number of medical conditions or events. A master search form is created as well as master maps. Providing access to displays of conditions or events is a patient/computer controlled system.

U.S. Pat. No. 6,063,026 to Schauss et al discloses a method and apparatus for use as a medical diagnostic system. It provides a first database containing disease indicators including human experience test results associated with the indicators. There is also provided a second database containing a plurality of drugs and the indicators associated with each drugs. Test results are input for an individual including specific diagnostic levels and comparing said specific levels of the individual with the indicator data in the database. The indicator presence levels are determined with preset specific levels associated with the individual. This information is compared to the indicator presence information contained in the second database to provide a detention of effects of the drugs in the individual.

U.S. Pat. No. 6,248,063 to Barnhill et al relates to an apparatus and process for diagnosing, screening or prognosticating diseases. In particular, data is obtained from a patient; the data is digitized; selecting which of the data are associated with a disease; scaling digitized values; performing tests to analyze the disseminating power of the data. Then using a trained computer to produce an output which may determine whether the patient has or is likely to have the disease.

U.S. Pat. No. 6,120,440 issued to Goknar discloses a computer controlled system for psychometric analysis and diagnosis based on a patient's reply to a series of questions. It is limited by the program and patient replies although at least one other patient is looked to for a comparison.

U.S. Pat. No. 6,053,866 to McLeod relates to a method of computerized psychiatric analysis based on patient answers to questions to establish a preliminary disorder identification which after analysis may suggest further questions to determine if additional disorders may exist.

U.S. Pat. No. 5,784,539 to Lenz is of interest and describes a computer system which may be applied to store and analyze medical data.

U.S. Pat. No. 5,935,060 to Iliff discloses to a system and method used by a patient and a computer to assess the existence or probability of a disease. In particular, the computer interviews the patient for a specific medical condition to then provide as an output a diagnosis. Again, it is a patient/computer controlled diagnosis for a condition.

Despite advances, according to the findings of a 2000 report, To Err is Human: Building a Safer Health Care System, by the Institute of Medicine, the medical arm of the National Academy of Sciences, an estimated 44,000+ Americans alone die each year as a result of medical errors, with an estimated cost between $17 billion and $29 billion, and as many as 98,000 Americans die each year from adverse medical events. Medication errors alone are estimated to account for over 7,000 deaths annually.

As published in The New England Journal of Medicine, Vol 330:1792(1994), the current computer-based diagnostic systems provide correct diagnoses opinions 52% to 71% of the time. Their conclusions were that the current systems should only be used by physicians who can identify and use the relevant information and ignore irrelevant information that was produced by existing systems.

### SUMMARY OF THE INVENTION

The present invention discloses an improved medical diagnostic system and computer means to its utilization that allows for analysis of available markers and other critically relevant indicia, provides direction in selecting additional markers and indicia for analysis, creates a profile for the patient being diagnosed, and compares this profile against known profiles for diseases and other medical conditions. Once potential diagnoses are found, the system presents a weighted list for a clinician to review and rule-in and rule-out tests or a need for markers to aid the clinician in reaching a final, accurate diagnosis, which can be explained to receive recommendations for treatment.

More particularly, the present invention relates a method and apparatus for a data system to receive quantitative diagnostic information and analyze the same to aid physicians in the diagnosis of disease or other maladies. It comprises: means to collect diagnostic information (data) on an ongoing basis provided by physicians and related medical specialists; filing the diagnostic information collected; comparing the collected information to input diagnostic information of a patient to determine potential diagnosis; reporting the same to the subscribing clinician with, if necessary, requests for additional input to refine the diagnosis and suggestion for treatment based on information stored and received. The database is refined based on confirmed diagnostic patterns submitted. Analyzing and storing input diagnostic information may be in graphical from to fingerprint a potential disease state and in developing an output form which is an automatic translation of diagnostic data in standard analysis format to define the disease state or what information is required to confirm a diagnosis by an iterative input and output. A diagnosis, if confirmed, and is used to treat the patient, is fed back to the system to expand its database refine its ability to provide an accurate data analysis and further diagnosis.

Patient data can be input from any location via the Internet or the like. It may be translated into standard graphical format; compared with database information on the patient, and possible diagnoses based on data are listed. The diagnostic choice is narrowed by suggesting additional diagnostic testing using a process of elimination and confirmation. A report of suggested diagnosis is generated with background information and other possible conformatory symptoms identified. The database may be updated with confirmatory information through inviting the physician for a final data input. The physician may be provided client database history of his patients.

### THE DRAWINGS

FIG. 1 is a block diagram illustrating the DIS overall flow of information according to the invention.

FIG. 2 is a block diagram illustrating the particular logic flow of patient diagnostic information use for the patient by subscribing clinicians.

FIG. 3A is a graphic illustration of established ranges for given blood or other sera components.

FIG. 3B is a graphic illustration of analysis of the constituents of an individual's sera (blood) relative to FIG. 3A to establish divergence from acceptable ranges.

### DETAILED DESCRIPTION

The Diagnostic Information System (DIS) of this invention provides clinicians (physicians and others in their control) information which aids in the diagnosis and treatment of diseases and other medical conditions. It comprises:
a) feeding to a living databank or database containing normal and abnormal diagnostic profiles a patients diagnostic profile,
b) comparing the patients diagnostic profile to relevant diagnostic profiles contained in the databank,
c) computing and delivering from the comparison a weighted list of potential diagnoses and treatments and recommendations for further rule-in/rule-out testing and marker requests to finalize diagnosis and treatment. The list of potential diagnoses is refined based on rule-in/rule-out tests and markers identified.

If the most probable diagnosis is confirmed by patient treatment, the confirmed information is added to the databank. Incorrect diagnostic information may also be added to aid in refining the indicia used in future analysis.

Data utilized and delivered may be transmitted through any format including the Internet. Data may be converted from digital to graphical for pattern comparison and missing data can be added for diagnostic pattern verification. In any event the fed indicia from verified diagnosis may be used to increase the size of or improve the databank. To this end, a gateway system may be and is preferably used to qualify indicia for entry into the living databank to maintain its integrity as a viable means for accurate diagnosis.

Definitions—As used herein and the claims the following have the stated meanings
a) Living Databank or Database—a data receptive bank or base having the characteristics of a living organism capable of digesting new information and growing in utility by adapting to both the advances of science and technology in the disciplines applied in diagnosis
b) Gating—a program for accepting only confirmed information relevant to a diagnosis in question to sustain the system's integrity
c) Rule-in/Rule-out—a yes/no evaluation of a bit of information in determining its relevance to a diagnosis under consideration
d) Weighted Diagnosis—of possible diagnoses reported as output, the relative probabilities of each to be the most likely
e) Clinician—physicians and/or medical professionals operating under their direction and control of the physician

The invention pertains to a marriage of the disciplines of medical science, clinical diagnostics and computer science while accommodating the exponentially increasing knowledge in these disciplines.

The diagnostic information is from any of a variety of sources. The sources include quantitative analysis of body fluids such as blood, saliva, and urine as well as quantitative and qualitative information obtained from x-rays, spinal taps, MRI, cat scans, ultrasound, biopsies, and the like. Every bit of relevant and critical information may be used in determining an analysis of possible diagnosis to be modified, if required or at all, in reporting probable diagnosis to the clinician as well as inputs desired to confirm or reject a diagnosis.

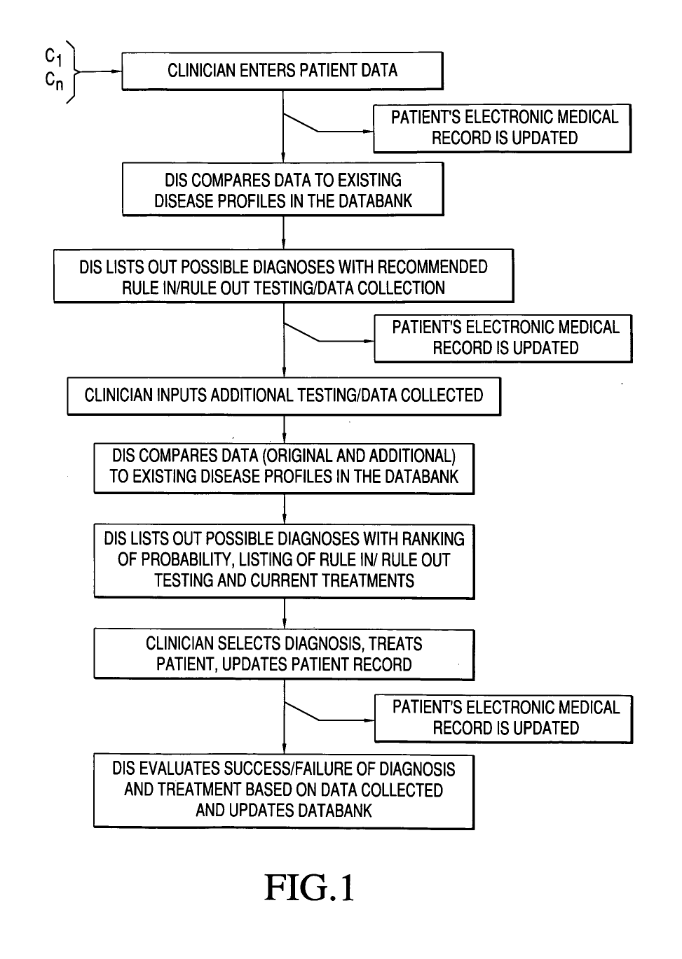

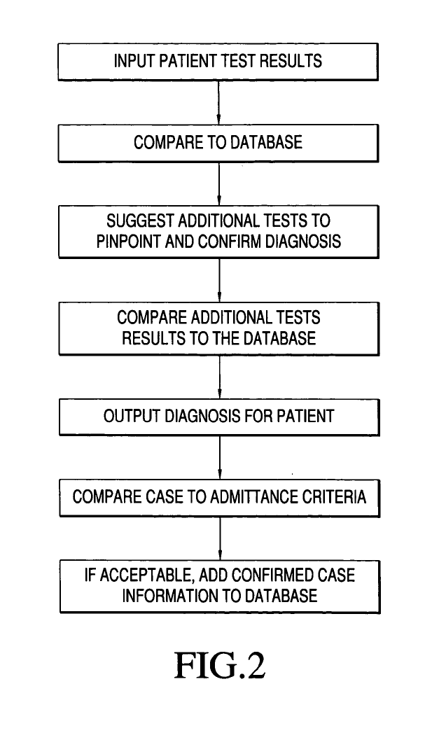

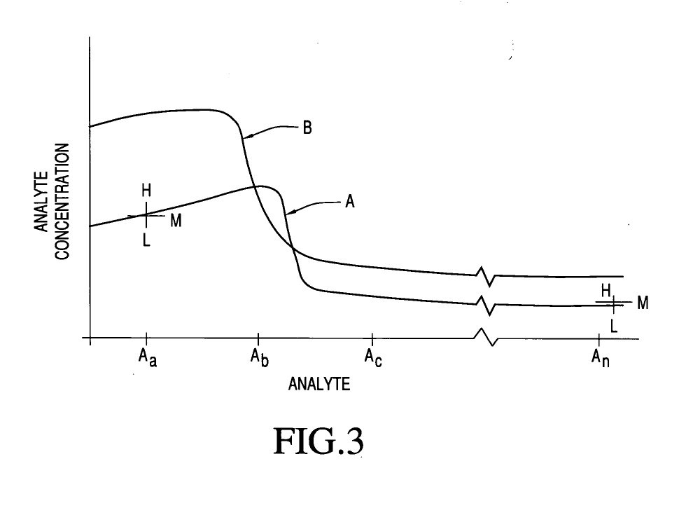

The general flow of developed information is shown in FIGS. 1 and 2 and a comparison of measured analyte concentrations as compared to standard concentrations in blood is shown in FIGS. 3A and 3B.

With reference now to FIGS. 1 and 2 developed information is input to be used to enhance the database and update patient information and form the basis for a patient profile which is compared to the profiles for similarity and possible matches contained in the databank. This results in an output of possible diagnoses, treatments and requests which the clinician uses to select and perform identified clinical tests an/or provide relevant marker information. This information is fed to the computer which establishes a diagnosis probabilities which is reported to and utilized by the clinician in treating the patient. This information is also used to enhance and update the patients medical record and modify the databank based on an evaluation of success or failure of suggested diagnosis and treatment based on data fed to and compared to data contained in the databank.

As indicated the present invention is directed to physician controlled exchange of diagnostic information to aid the physician in forming an opinion about a disease state and how to treat it. FIG. 1 displays a generalized block diagram for the flow of information, FIG. 2 is a more specific block diagram As to each, the initial input is the existing median and recognized parameters of measurable components as shown in FIG. 3A for sera (blood) and other indicia. The comparison is used to determine the possibility of a disease and means to treat it.

In particular FIGS. 3A and B illustrate test results obtained from a patient against standardized analysis. There is shown in FIG. 3A the limits and median of the known information to date.

FIG. 3B depicts the results of an individual's test to establish the deviance from the standard shown in FIG. 3A.

The objective is to find by proposed treatment an input of data to reshape FIG. 3B to FIG. 3A or compress or expand the ranges shown in FIG. 3A standard to be after integrate the patient's information to confirm, reject, or better analyze the existence or possibility of a disease or other malady.

It is controlled by physicians or those under their direction and control and designed to be released to participating physicians or those under their addition and control. As indicated, the procedure of this invention is physician controlled. It may be direct control or through a controlled person such as another, nurse, pharmacist, clinician or the like.

With reference again to FIGS. 1 and 2 there is shown the flow of information in and out of the database. The database is fed by physicians (P1, P2 etc) or those under their direction and control.

Data received is compared to established sera ranges such as shown in FIG. 3A with normal limits (H for high, L for low) of acceptability. FIG. 3A also shows an established median (N).

The physician, given a patient's chemistry profile, e.g. FIG. 3B, uses measures to bring the patient within the limits of FIG. 3A. The measures taken if utilized to success may be input into the database to confirm or refine information contained in the database.

This information is used to report the effects of treatment to participating physicians who have confirmed a deviation from established limits using gathered information and existing ranges. The collected information is used to change the database based on confirmed diagnostic patterns; fingerprint a disease state and develop translatable input and output forms which are an automatic translation of input data into a accepted diagnosis format whether digital or graphical.

For example, a patient exhibiting chest pains may cause the clinician to initially conclude the possibility of a heart attack, indigestion, stroke or like possibilities. By feeding developed information to the system such as panel results and other relevant information, the system will provide an initial range of possibilities and means to remedy the condition. Additional tests and other information are supplied to the clinician. By an interactive exchange between the clinician and the system, the most likely diagnosis can be arrived at. The system will propose, based on information received, the probable treatments to be administered. If the treatments are worthwhile, such information may be added to refine the databank. If negative, it may also be added to refine the databank.

The system is clinician/computer controlled and can respond to data input as fast as the computer contained information will allow. No bit of information is irrelevant or to be ignored. If confirmed to have diagnostic value it can be input or stored in the databank for fixture use.

While receiving information in digital format, the system convert to graphical format and produce an output in graphical and/or digital format. Either can instruct the clinician as to information desired or needed for a final diagnosis. It will provide an initial means of treatment and by an exchange of information to and from the clinician a final report of the diagnosis and method of its treatment. Because the iterative exchange of information is computer controlled the speed of results is limited only by time, that is the time required to feed information, amend information and report to the clinician results as of date. How the clinician provides and receives data is controlled by the clinician independent of analysis and recommendations reported. The reality is that by exchange of information the most likely diagnosis and its treatment can be defined.

Besides patients, these to be benefited are other physicians, drug manufacturers and insurers who assess the cost of medical treatment for individuals.

Patient data can be input from any location via the Internet or the like. It is automatically translated into sand digital or as desired, graphical format; compared with database information and a possible diagnosis based on input data is listed. The diagnostic choice is narrowed by suggested additional diagnostic testing using a process of elimintion and confirmation. A report of suggested diagnosis is generated with background information and other possible confirmatory symptoms identified. The database is updated with confirmatory information through inviting the physician for a final data input. The physician may provide and may be provided client database history of his patients.

The types of analysis used include evaluation of all measurable substance in the fluids such as urine and blood ranging from Fa to Fn on FIGS. 3A and 3B to more specific markers, as for instance, diabetes factors, arthritic factors and the like.

In all instances, the clinician is in control and for cooperation may be allowed to obtain outputs for his patients as consideration for patient inputs to the database. The basic data and patient data may be developed using known standard chemicals or procedures as described for instance in Clinical Chemistry Journal Supplement, Effect of Disease on Clinical Laboratory Tests, Clin Chem, Vol 26(4), 1980; Current Diagnosis/Conn's 7^{th} ed, French's Index of Differential Diagnosis, 12^{th} ed, Manual of Emergency Medicine, Manson's Textbook of Tropical Diseases, Conn's Current Therapy 3^{rd} ed., Clinical Decision Levels for Lab Test, 1^{st} ed., Internal Medicine Textbook, 2^{nd} ed., The Merck Manual, 17^{th} ed., Current Diagnosis, 9^{th} ed., each incorporated by reference.

The type of the diagnostic information system of this invention greatly improves the percentage of correct diagnoses based on utilization of profiles based on real cases which are updated as new findings occur, providing a broad base of diagnostic information for use in forming and expanding the diagnostic profiles. Safe guards may be and preferably are built into the system throughout to alert physicians of potential errors.

In addition to a reduction in diagnosis which in itself, can save lives and reduce the costs associated with lost lives, the diagnostic information system of this invention can reduce the overall cost of healthcare by reducing the time to accurate diagnosis.

Diagnostic methods used to identify medical conditions rely on the identification and utilization of quantifiable markers provided by clinicians. Such markers also include subjective markers such as physical symptoms, family histories, x-ray, MRI data and the like. Where this type of data does not readily lend itself to computer application, their use is a must a their value in diagnostics is undeniable. This system can translate such “soft” data into hard numeric data for computer application and ultimately to graphical information as desired.

The system of this invention provides physicians with data to aid in the diagnosis and treatment of disease and other medical conditions. Flowcharts of the system are illustrated in FIGS. 1 and 2. The method by which it operates is to:
1. collect qualified diagnostic information on an on-going basis from participating clinicians and adding such information to modify existing diagnostic profiles;
2. compare a patient's diagnostic profile against disease and other medical condition profiles stored in the system's database;
3. compute a weighted list of profile diagnoses and treatments with further rule-in/rule-out testing/marker recommendations;
4. refine the list of potential diagnoses based on input of suggested rule-in/rule-out testing/marker information; and
5. allow diagnostic profiles to be confirmed and added to the databank.

The diagnostic information system contains many highly innovative aspects. They may include:
a) the ability to convert digital data into a graphical pattern for diagnostic comparison;
b) identification and listing of missing data for pattern completion or verification; and
c) means to increase the size of the databank by using verified diagnostic patterns added to the databank.

In addition, the system preferably includes means to create a software gating system to qualify information obtained for inclusion in the databank. This method of qualification keeps the integrity of the “living database” viable for accurate diagnosis on an on-going basis.

In addition to receiving test data, the system may, as required, suggest additional diagnostic information needed to increase the probability of an accurate diagnosis. The additional information is to either rule-in or rule-out the most probable diagnoses. Probability factors are generated by recent databank entries, history and demographics of patient, initial test that entered into the system by the clinician, and other relevant information. Also, the probability of a rapid, accurate diagnosis is greatly increased by the living databank. As the databank grows, in real time, the clinician will be able to see trends and increased likelihood of diseases or other medical conditions based on markers such as demographics, such as age, geographical location, family history, genetic predisposition and the like.

The accuracy of the diagnosis is enhanced by the gating system which maintains the integrity of the databank. Diagnoses requires confirmation based on successful treatment and other proven methods of verification prior to incorporation into the system databank.

Significant elements of the system include digital translation of qualitative data; an auto feedback gating software that is able to accept only confirmed diagnosed cases or information and acts to marry the disciplines of medical science, clinical diagnostics and computer science; and accommodates the exponentially increasing knowledge base in these disciplines.

The minimum results from the practice of the invention are better enabling computer assisted diagnosis, reduce the time required for diagnosis, reduce the cost of diagnosis, and increase the accuracy of diagnosis.

This creates a quantum leap in transforming the current “art” of diagnostics into more of a scientific discipline and one that is less dependent on the qualitative soft data interpretation and limited by the individual knowledge base of the clinician, and is more based on the collective knowledge base of the medical community. It augments the physician's curt database and greatly expands it. It may employ a voice signature “squawk box” used in the physicians examining room and electronic pens/pads connected to a computer system that is able to take the physician's observations, issue laboratory orders, process test results and provide the physicians with the patient's medical records, diagnosis, suggests available therapy and logic for arriving at the results and suggested course of action presented. The knowledge base increase in the scientific disciplines is utilized to expand the database and increase the sophistication of data usage, as well as computations employed. Therefore the convenience and accuracy of diagnosis, medical record keeping and the effectiveness of therapeutic processes are in the living database continuously enhanced.

Perhaps the greatest value of this system is to increase the scientific knowledge base in the field of medical diagnostics and to keep on increasing this knowledge base continuously in the future and make it readily available to the medical disciplines to reduce human suffering. But even as important is that it provides the medical communities of physicians instant access to a database that previously took long hours, days or weeks to research. In addition the system databank is based on confirmed diagnosed cases, not theories or conjectures. Because of this, complete information becomes available to all clinicians not just a few who can or will spend the needed research time to follow up cases.

The following illustrates the practice of this invention.

### EXAMPLE

A patient presents himself to his clinician. He is an African-American in his mid-30s. He has had a sore throat for more than 24 hours. The clinician collects relevant data for entry into the diagnostic information system: sore throat, pulse rate 120 beats per minute, blood pressure 115/75, audible respiration, trouble with swallowing, fever (102 degrees F.), non-smoker, moderate drinker, no medications, no allergies.

Computer returns possible diagnoses:
Retropharyngeal or peri-tonsillar infections
Infectious mononucleosis
Diphtheria
Ludwig's angina
Epiglottitis
Allergic drug reactions
Foreign bodies
Tumors or trauma to the larynx
Inhalation or aspiration of toxic chemicals

Physician selects epiglottitis. The computer recommends neck and chest examination and a CT scan for rule-in/rule-out refinement. Physician performs the tests and enters data into the computer system. The CT displays thickening of epiglottitis, aryepiglottic folds false and true vocal cords. Chest examination is unremarkable, except for transmitted sounds. Aside from the marked sinus tachycardia, the cardiovascular examination is normal. Additionally, anterior tenderness in the neck is found.

The computer returns epiglottitis as the diagnosis and recommended treatment is listed as intubation as needed and antibiotic treatment. Typically, second or third generation cephalosporins are used.

Physician provides treatment to the patient. Treatment is successful. Physician updates the profile with successful treatment. The profile is added to the patients profile and the system database.

1. A method for computerized determination of an abnormal medical condition in a human patient which comprises:
a) inputting, to a living databank containing a plurality of diagnostic profiles of normal and abnormal medical conditions, a diagnostic profile of at least one patient as supplied by at least one clinician;
b) comparing the input patient diagnostic profile to the diagnostic profiles contained in the living databank;
c) computing and reporting to the clinician based on the comparison a weighted list of possible abnormal medical conditions and means of treatment and, in order, suggestions for further diagnostic tests and markers;
d) further computing and reporting, on an iterative basis, and based on all clinician responses to suggested further diagnostic tests and markers a refined possible diagnosis and treatment;
e) continuously refining the diagnostic living databank utilizing clinician input of confirmed diagnosis and treatment.

2. A method as claimed in claim 1 in which abnormal diagnosis or treatment are input to the living databank to refine the diagnostic profiles contained in the living databank.

3. A method as claimed in claim 1 in which input information is processed by a gating program to accept only information confirmed and relevant to the diagnosis in question.

4. A method as claimed in claim 1 in which input digital data for a patient is converted to graphical format at least for the purposes of the comparison.

5. A method as claimed in claim 1 in which any output is presented in formats selected from verbal, written, digital, graphical and a combination thereof.

6. A method as claimed in claim 4 in which any output is presented in formats selected from verbal, written, digital, graphical and mixtures thereof.

7. A method as claimed in claim 1 in which the patient profile and other relevant information are input from remote sources.

8. A method as claimed in claim 1 in which the computed results are transmitted to a remote receiver.

9. A method as claimed in claim 7 in which the computed results are transmitted to a remote receiver.

10. A method as claimed in claim 1 which inputs are received from multiple clinicians and reports are made to multiple clinicians.

11. A method as claimed in claim 10 in which the computed results are transmitted to at least a remote receiver.

12. A method for computerized determination of an abnormal medical condition in a human patient which comprises:
a) inputting, to a living databank containing a plurality of gated diagnostic profiles of normal and abnormal medical conditions, a gated diagnostic profile of at least one patient as supplied by at least one clinician;
b) comparing the gated input patient diagnostic profile to the relevant diagnostic profiles contained in the living databank;
c) determining on a rule in/rule out basis a weighted list of possible abnormal medical conditions and means of treatment and requests for further diagnostic rule in/rule out tests and markers;
d) further computing on gated basis and reporting, on an iterative basis, and based on all clinician responses to suggested further diagnostic tests and markers a refined possible diagnosis and treatment;
e) refining the diagnostic living databank utilizing clinician input of confirmed diagnosis and treatment.

13. A method as claimed in claim 12 in which abnormal diagnosis or treatment are input to the living databank to refine the diagnostic profiles of the living databank.

14. A method as claimed in claim 12 in which input digital data for a patient is converted to graphical format at least for the purposes of the comparison.

15. A method as claimed in claim 14 in which any output is presented in formats selected from verbal, written, digital, graphical and combinations thereof.

16. A computer system for diagnosis of diseases and other human maladies which comprise:
a) living databank programmed to receive and retain on an ongoing basis clinical diagnostic and marker information input by a clinician;
b) means to form from input diagnostic information profiles of normal and abnormal medical conditions;
c) means to receive diagnostic and marker information to form a profile of the patient;
d) means to compare the formed profile of the patient to contained normal and abnormal profiles;
e) means to compute a weighted average of possible diagnosis and treatments and determine needs for fir diagnostic tests and markers;
f) means to report to the clinician the results of the computed weighted average;
g) means to receive and respond to responses from clinicians of further clinical test and markers to refine the diagnosis and methods for treatment; and
h) means to update the living databank from feedback on the results of step (g).

17. A computer system as claimed in clan 16 which is adapted to receive diagnostic and marker information from a remote transmission means and deliver responses and requests to the remote receiver.

18. A computer system as claimed in claim 16 including gating means to retire information supplied to the databank to receive or reject information relevant to the databank.

19. A computer system as claimed in claim 16 including means to receive and transmit information by means selected from the group consisting of oral, digital, graphical sad combinations thereof.

20. A computer system as claimed in claim 16 which includes means to cornet received information to graphical format and transmit information in graphical and digital format.
